(19) United States
(12) Patent Application Publication
(10) Pub. No.: US 2008/0058729 A1
(43) Pub. Date: Mar. 6, 2008

(54) VASCULAR ACCESS HARNESS FOR SMALL LABORATORY ANIMALS

(75) Inventors: Michael H. Loughnane, Lafayette Hill, PA (US); Bernd Fisher, Pottstown, PA (US); Andrew Jacobson, San Antonio, TX (US)

Correspondence Address: FOLEY AND LARDNER LLP;SUITE 500, 3000 K STREET NW, WASHINGTON, DC, 20007, US

(73) Assignee: INSTECH LABORATORIES, INC.

(21) Appl. No.: 11/847,591

(22) Filed: Aug. 30, 2007

Related U.S. Application Data
(60) Provisional application No. 60/841,530, filed on Sep. 1, 2006.

(51) Int. Cl. A61M 39/10

(52) U.S. Cl. 604/175

(57) ABSTRACT

A device and method for accessing a vascular system of small laboratory animals. A port unit is configured to connect to an indwelling catheter of the animal. The port unit is secured to the animal by a harness block. At least one elastomeric band secures the harness block around the animal. A mating unit is configured with a cannula that pierces a septum at the top of the port unit. The catheter is connected to a conduit via a spring tether and allows a test substance to pass to the animal's vascular system from the conduit via a pathway through the cannula to the port unit and to the indwelling catheter. The septum prevents both forward and backward leakage and contamination of the indwelling catheter even after repeated connections and disconnections.

### FIELD OF THE INVENTION

The present invention relates generally to the field of catheterized laboratory animals. In particular, the present invention relates to a vascular access harness for use with laboratory animals with indwelling catheters.

### BACKGROUND OF THE INVENTION

The following description of the background of the invention is provided simply as an aid in understanding the invention and is not admitted to describe or constitute prior art to the invention.

In order to provide a continuous fluid connection to an awake, freely-moving laboratory animal for drug infusion, fluid sampling, pressure monitoring and other related procedures, it is necessary to implant a catheter. Researchers often purchase laboratory animals with indwelling catheters already implanted, locked with an anticoagulant to prevent clotting and coiled under the skin. When an animal arrives at the user facility, the user typically opens the closed wound, pulls out the catheter coil, closes the wound again, and connects the indwelling catheter to external tubing which runs through a protective spring tether, on to a swivel to prevent tangling and ultimately to a pump or monitor.

Current techniques include harnesses or jackets to anchor the tether to the animal and friction-fit couplers (Instech Laboratories, Inc., Plymouth Meeting Pa.) or standard Luer fittings (Strategic Applications, Inc., Libertyville, Ill.; AVA Biomedical Inc., Winnetka, Ill.) to connect the indwelling catheter to external tubing. When such devices are disconnected, the indwelling catheter is open to the atmosphere. Under these conditions, it is possible for microorganisms to enter the open end and cause infection. In addition, blood can flow back into the tip of the indwelling catheter, possibly leading to clot formation.

Accordingly, there is a need for a catheter connection and disconnection device that reduces the time and effort spent preparing a catheterized laboratory animal for testing and reduces the risk of infection and clot formation in a catheter attached to the laboratory animal.

### SUMMARY OF THE INVENTION

In view of the above described limitations, it is an objective of the present invention to provide a catheter connection and disconnection device for small laboratory animals. The Vascular Access Harness (VAH) of the present invention allows for fast, simple, and aseptic connection and disconnection, with respect to an infusion tether, of a catheterized small laboratory animal, such as a mouse, rat, guinea pig, or rabbit.

In general terms, one embodiment of the vascular access harness system can include a miniature external port that has a penetrable, self-sealing septum, housed in a harness which the animal wears. A short connector tube can extend from the bottom of the port to attach to the animal's indwelling catheter on the underside of the harness. Elastomeric tubing, which forms belly bands, can hold the harness in place around the animal.

The mating connector can attach to a spring tether, and can house a septum-piercing needle in a protective shroud, which fits over the top of the external port and preferably is held in place by friction. A secondary catheter, attached to the rear of the septum-piercing needle, can lead up to an infusion device, optionally through a fluid swivel to allow free animal movement.

According to another embodiment, a method for accessing the vascular system of a small animal includes the steps of attaching a harness block to a small laboratory animal using at least one elastomeric bands to wrap around the animal's body and connecting an access port of the harness block to an indwelling catheter of the animal.

The method for accessing the vascular system of a small animal also includes the steps of connecting a mating connector to the access port of the harness block, piercing a septum of the harness block with a cannula to form a fluid line from a conduit through the access port to the indwelling catheter of a animal and transferring an infusion source through the fluid line to the indwelling catheter of the animal.

It is to be understood that both the foregoing general description and the following detailed description are exemplary and explanatory only, and are not restrictive of the invention as claimed.

### BRIEF DESCRIPTION OF THE DRAWINGS

The accompanying drawings, which are incorporated in and constitute a part of this specification, illustrate exemplary embodiments of the invention and together with the description serve to explain principles of the invention.

FIG. 1 is an exploded view of the port unit and mating connector of the vascular access harness device according to one embodiment.

FIG. 2 is a cross-sectional view through a center line of the device with the port unit and mating connector disconnected according to one embodiment.

FIG. 3 is a cross-sectional view through a center line of the device with the port unit connected to the mating connector according to one embodiment.

FIGS. 4(a)-(c) are images of the device in disconnected and connected states according to one embodiment.

FIG. 5 is a protected locking and flushing assembly, according to one embodiment.

### DETAILED DESCRIPTION

Presently, preferred embodiments of the invention are illustrated in the drawings. Embodiments of the present invention will be described below with reference to the accompanying drawings. It should be understood that the following description is intended to describe exemplary embodiments of the invention, and not to limit the invention. An effort has been made to use the same or like reference numbers throughout the drawing to refer to the same or like features.

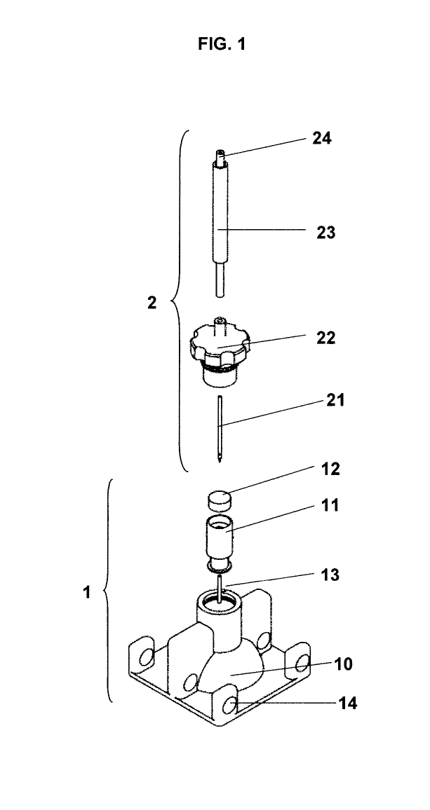

According to a preferred embodiment of the present invention, a harness device, as shown in FIGS. 1-3, comprises a port unit 1, which can further comprise a harness block 10, preferably composed of a molded, elastomeric material that is conformed to a body surface of a catheterized small animal. The port unit 1 can further comprise an access port 11 with a first end and a second end. The access port can be configured to interconnect at the first end with the external end of the animal's indwelling catheter (not shown) through a connector tube 13. The access port 11 can be sealed at its second end by a septum 12. The septum 12 is preferably composed of a material, such as silicon rubber, that can be perforated by a septum-piercing needle 21 multiple times, without developing a significant leak.

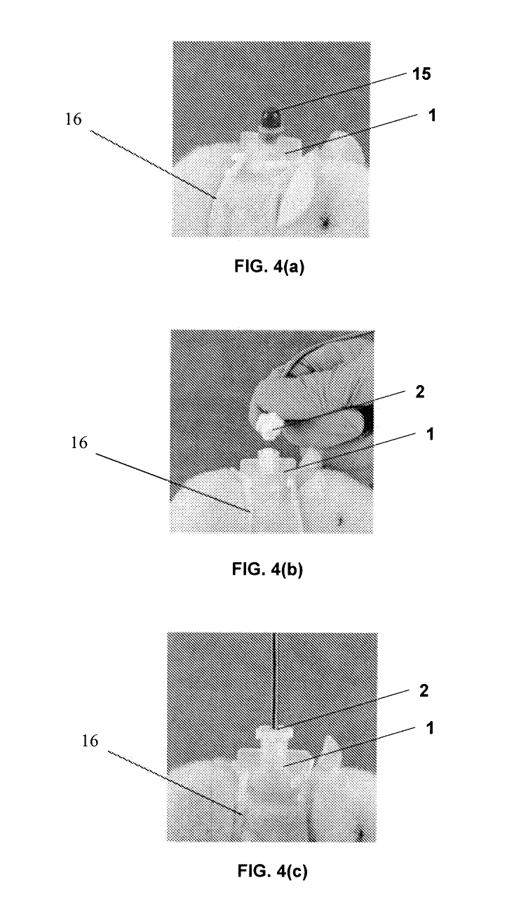

In addition, the inventive harness device comprises at least one non-rigid band 16 (shown in FIGS. 4(a)-4(c)), typically of an elastomeric material, which effect a high-friction fit to the harness block 10 and can be adjusted to the animal. Preferably, two bands 16 are secured to the harness block 10. The bands 16 fit through holes 14 in harness block 10. Thus, the bands 16 can secure the port unit on the animal's body surface, as shown in FIGS. 4(a)-(c). Preferably, the port unit 1 can be sealed at the second end of access port 11 by a protective cap 15, composed of a molded elastomeric or other suitable material.

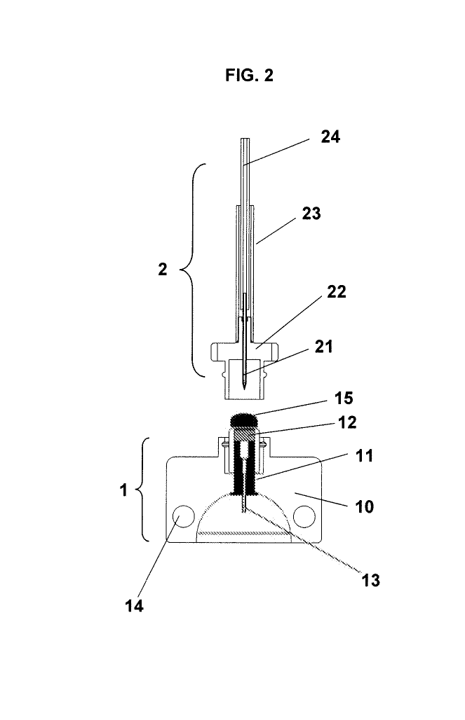

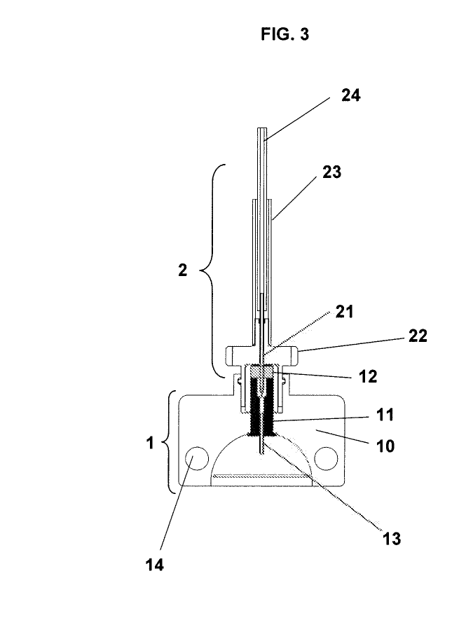

In use during a laboratory, research or other like testing operation, the vascular access harness of the invention further includes a mating connector 2, as indicated in FIGS. 2-3. The mating connector can comprise a housing or tether cap 22, which is configured to engage the second end of the port unit. A cannula 21, preferably in the form of a septum-piercing needle, is positioned within the housing 22 such that, upon engagement of the housing 22 with the port unit 1, the cannula 21 pierces the septum 12. A conduit, such as a catheter 24, is in fluidic communication with the cannula 22.

The conduit or catheter 24 is optionally positioned within a protective spring tether 23 for simple and easy movement of the mating connector 2. The protective spring 23 can optionally be connected to the conduit 24 via the spring tether 23. Accordingly, upon engagement of the port unit 1 and the mating connector 2, as illustrated in FIG. 3, a fluid, which may contain a test substance or an infusion source (not shown), can pass to the access port 11 via a pathway defined by the conduit 24 and the cannula 21. The test substance or infusion source can be connected to the conduit 24.

To connect the mating connector 2 to the port unit 1, the user removes the protective cap 15 and places the mating connector 2 over the top of the external port 11. The cannula 21, preferably a septum piercing needle, then pierces the septum 12. The mating connector 2 is preferably held in place by friction. Little or no retrograde flow occurs, when the mating connector 2 is removed from the harness 10 because of the function of the septum seal 12.

Once connected the user may infuse a test substance through the conduit or catheter 24. The test substance can travel through a path via the conduit or catheter 24, the cannula 21, the access port 11, and the connector tube 13 to enter the animal's indwelling catheter.

FIG. 4(b) shows the protective cap 15 removed and the mating connector 2 prepared to connect to the port unit 1. FIG. 4(c) shows the mating connector 2 connected to the port unit 1. If an animal vendor has installed the harness of the present invention as shown in FIGS. 4(a)-(b), the user can merely remove the protective cap 15, swab the top of the septum with disinfectant, and then plug in the mating connector 2, whereby a test substance, such as a infusion source, can be transferred to the animal through its indwelling catheter. Furthermore, the vascular access harness of the present invention allows the animal to be disconnected and weighed without interference of the mating connector 2.

The vascular access harness of the present invention allows the user to easily flush the indwelling line and introduce a “locking solution” to further prevent clotting at the tip of the indwelling catheter. When the animal is not connected to the mating connector 2, the protective cap may be replaced to prevent bacterial contamination of the access port 11.

In the form of a kit, with appropriate instructions of their deployment, the port unit 1 and mating connector 2 may be provided separately to the user.

According to a preferred method of the present invention, the indwelling catheter (not shown) is never disconnected. A small laboratory animal, such as a mouse, rat, guinea pig, or rabbit, may be purchased with an indwelling catheter or the user may insert the catheter into the animal surgically. In either case, the user places a harness block 10 that secures around the animal with at least one non-rigid band 16 as shown in FIGS. 4(a)-4(c). According to this method, an access port 11 engages with the indwelling catheter of the animal to create a fluid line to the animal's vascular system.

The user removes a protective cover 15 from the access port unit and then can engage a mating connector 2 to the access port unit 1 to create fluid communication between an infusion source and the indwelling catheter. The mating connector contains a cannula 21 that pierces a septum 12 at the top end of the access port unit 11. The mating connector can transmit fluid, such as a test substance, via the a catheter 24 that is in fluid communication with the cannula 21 and the access port unit 11. When the mating connector is removed from the access port unit 1, the septum 12 prevents leakage and can be recovered with the protective cover 15.

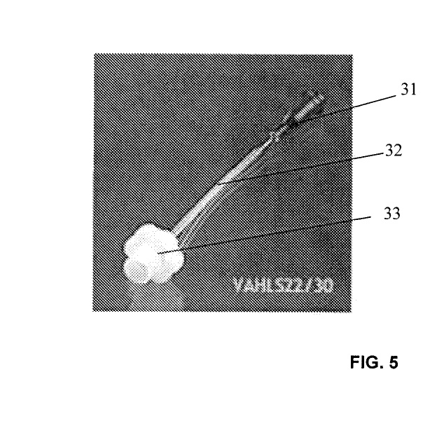

It is sometimes necessary to flush and relock the catheters when animals have been disconnected for a period of time. With the VAH, the access port 11 can be accessed with a syringe using the same type of septum piercing needle 21. In situations where highly infectious agents are being tested, as shown in FIG. 5, a shrouded needle assembly 31, as is used on the end of the tether 23, can be attached to a short length of tubing 32 and then to a fitting 33 to attach to the syringe (not shown). The needle is thereby protect and prevents accidental injury to the technician.

The present invention has several advantages. The VAH can be installed at the time of catheter implantation and filled with locking solution in the sterile operating environment. No longer is it necessary for the user to manipulate the wound or couplings. Chances for infection at the wound site as well as possible microorganism contamination of the open catheters are reduced. The animal is ready for attachment to the external system immediately after swabbing the septum with disinfectant. Bleed-back, or retrograde flow, into an open catheter is reduced by virtue of the sealed septum, reducing the chances for a catheter tip clotting. Further, when animals are used for periodic vascular access, the device and method of the invention greatly simplify the process. For example, the VAH makes it easier to disconnect the animal for periodic weighing during a study.

The foregoing description of a preferred embodiment of the invention has been presented for purposes of illustration and description. It is not intended to be exhaustive or to limit the invention to the precise form disclosed, and modifications and variations are possible in light of the above teaching or may be acquired from practice of the invention. The embodiment was chosen and described in order to explain the principles of the invention and as a practical application to enable one skilled in the art to utilize the invention in various embodiments and with various modification are suited to the particular use contemplated. It is intended that the scope of the invention be defined by the claims appended hereto and their equivalents.

Given the disclosure of the present invention, one versed in the art would appreciate that there may be other embodiments and modifications within the scope and spirit of the invention. Accordingly, all modifications attainable by one versed in the art from the present disclosure within the scope and spirit of the present invention are to be included as further embodiments of the present invention. The scope of the present invention is to be defined as set forth in the following claims.

What is claimed is:

1. A vascular access harness device for a small laboratory animal, comprising:
a port unit further comprising a harness block, which conforms to a body surface of said animal;
an access port with a first end and a second end, wherein said access port is configured (a) to interconnect at said first end with an indwelling catheter of the animal and (b) is sealed at said second end by a septum; and
at least one non-rigid band that interacts with said harness block, such that said band can secure said port unit on said body surface of the animal.

2. The device according to claim 1, further comprising a protective cap engaged with the second end of the access port on top of the septum.

3. The device according to claim 1, wherein the access port further comprises a coupling tube between the first end of the access port and the indwelling catheter of the animal.

4. The device according to claim 1, wherein the harness block is custom molded to fit the size of the laboratory animal.

5. The device according to claim 1, further comprising:
a mating connector comprising:
a housing, which is configured to engage said second end of said port unit;
a cannula that is positioned within said housing such that, upon engagement of said housing with said port unit, said cannula pierces said septum; and
a conduit in fluidic communication with said cannula such that, upon said engagement, a fluid can pass to said access port via a pathway defined by said conduit and cannula.

6. The device according to claim 5, wherein said mating connector is engaged with said second end of said port unit.

7. The device according to claim 5, further comprising a protective spring tether enclosing the conduit.

8. The device according to claim 5, further comprising an infusion source connected to the conduit, wherein a test substance can pass from said infusion source to said indwelling catheter via a pathway defined by said conduit, port and cannula.

9. A method for accessing the vascular system of a small animal comprising:
attaching a harness block to a small laboratory animal using at least one elastomeric band to wrap around the animal's body; and
connecting an access port of the harness block to an indwelling catheter of the animal.

10. The method of claim 9, further comprising:
connecting a mating connector to the access port of the harness block;
piercing a septum of the harness block with a cannula to form a fluid line from a conduit through the access port to the indwelling catheter of a animal; and
transferring an infusion source through the fluid line to the indwelling catheter of the animal.

11. The method of claim 9, wherein the indwelling catheter is surgically implanted into the animal.

12. The method of claim 9, wherein the small laboratory animal is a mouse, rat, guinea pig, or rabbit.
